(19) United States
(12) Patent Application Publication
(10) Pub. No.: US 2015/0327772 A1
(43) Pub. Date: Nov. 19, 2015

(54) PHOTOACOUSTIC APPARATUS

(71) Applicant: CANON KABUSHIKI KAISHA, Tokyo (JP)

(72) Inventor: Yoshitaka Baba, Tokyo (JP)

(21) Appl. No.: 14/710,423

(22) Filed: May 12, 2015

(30) Foreign Application Priority Data
May 14, 2014 (JP) 2014-100853

(51) Int. Cl. A61B 5/00

(52) U.S. Cl. CPC A61B 5/0095; A61B 5/7225; A61B 2560/0475; A61B 2562/046

(57) ABSTRACT

A photoacoustic apparatus includes a light source, a plurality of receiving elements that receive a photoacoustic wave generated as a subject is irradiated with light emitted from the light source and output time-series reception signals, a signal data acquisition unit that generates reception-signal data based on the time-series reception signals and store the reception-signal data, and an information acquisition unit that acquires information on the subject based on the reception-signal data stored in the signal data acquisition unit.

### BACKGROUND

1. Field

Aspects of the present invention generally relate to photoacoustic apparatuses that acquire subject information with the use of photoacoustic effects.

2. Description of the Related Art

In the medical field, active researches are being made on optical imaging apparatuses that irradiate a subject, such as a living body, with light emitted from a light source, such as a laser, and that form images from information on the inside of the subject acquired on the basis of the light incident on the subject. Examples of such an optical imaging technique include photoacoustic imaging (PAI). In photoacoustic imaging, a subject is irradiated with pulsed light emitted from a light source; acoustic waves (typically, ultrasonic waves) emitted from the subject's tissues that have absorbed the energy of the pulsed light which has propagated and been diffused in the subject are received; and an image is formed from subject information on the basis of received signals.

Specifically, a difference in absorptance of the optical energy between a target site, such as a tumor, and other tissues being utilized, elastic waves (photoacoustic waves) emitted when a target site that has absorbed the irradiated optical energy momentarily expands are received by a probe. The received signal is mathematically analyzed, and thus the information on the inside of the subject, in particular, an initial sound pressure distribution, an optical energy absorption density distribution, an absorption coefficient distribution, and so on can be obtained. Such pieces of information can be used to quantitatively measure a specific substance inside the subject, such as the oxygen saturation in blood. In recent years, preclinical studies in which angiograms of small animals are obtained by using the above-described photoacoustic imaging and clinical studies in which the principle of the photoacoustic imaging is applied to the diagnosis of breast cancers have actively been carried out (“Photoacoustic Tomography: In Vivo Imaging from Organelles to Organs,” Lihong V. Wang, Song Hu, Science, 335, 1458 (2012)).

U.S. Pat. No. 5,713,356 describes an apparatus in which thoracic tissues are irradiated with electromagnetic waves and a probe receives photoacoustic waves generated as the thoracic tissues are irradiated with the electromagnetic waves and outputs a reception signal, which is then stored in a memory. In addition, U.S. Pat. No. 5,713,356 indicates that an image of the thoracic tissues is formed by using data of the stored reception signal.

In an apparatus such as the one described in U.S. Pat. No. 5,713,356, a reception signal outputted from a transducer needs to be stored in a memory. In the meantime, it is desirable to reduce the amount of data of a reception signal to be stored in the memory.

### SUMMARY

According to an aspect of the present invention, a photoacoustic apparatus includes a light source, a plurality of receiving elements configured to receive a photoacoustic wave generated as a subject is irradiated with light emitted from the light source and output time-series reception signals, a signal data acquisition unit configured to generate reception-signal data based on the time-series reception signals and store the reception-signal data, and an information acquisition unit configured to acquire information on the subject based on the reception-signal data stored in the signal data acquisition unit. The signal data acquisition unit determines a sampling frequency based on a distance from a specific position to a surface of the subject, samples the time-series reception signals at the sampling frequency so as to generate the reception-signal data, and stores the reception-signal data.

Further features of the present disclosure will become apparent from the following description of exemplary embodiments with reference to the attached drawings.

### BRIEF DESCRIPTION OF THE DRAWINGS

FIG. 1 illustrates a configuration of a photoacoustic apparatus according to an exemplary embodiment.

FIG. 2 illustrates connections among a computer and other components according to an exemplary embodiment.

FIG. 3 is an illustration for describing a method for determining a sampling frequency according to an exemplary embodiment.

FIG. 4 illustrates a flow of operations of a photoacoustic apparatus according to an exemplary embodiment.

FIG. 5 illustrates a computer according to an exemplary embodiment in detail.

FIG. 6 illustrates an example of a sampling frequency according to an exemplary embodiment.

FIG. 7 illustrates a sampling sequence according to an exemplary embodiment.

### DESCRIPTION OF THE EMBODIMENTS

Hereinafter, exemplary embodiments will be described with reference to the drawings. It is to be noted that the dimensions, the materials, and the shapes of components described hereinafter and the relative arrangement among the components are to be modified, as appropriate, in accordance with the configuration or various conditions of an apparatus to which the exemplary embodiments are applied, and these exemplary embodiments are not seen to be limiting.

For example, in photoacoustic imaging, it is effective to generate an image that is based on subject information on the basis of reception signals of photoacoustic waves that contribute significantly to an increase in image quality, in order to acquire a high-quality image in photoacoustic imaging.

However, when photoacoustic waves are received as described in U.S. Pat. No. 5,713,356, photoacoustic waves that do not contribute significantly to an increase in image quality may also be received. Examples of the photoacoustic waves that do not contribute significantly to an increase in image quality include, among photoacoustic waves generated inside a subject, a photoacoustic wave of a frequency component that has attenuated to a great extent while propagating through the inside of the subject. Even when a reception signal of such a photoacoustic wave of a frequency component that has attenuated to a great extent is used, the reception signal does not contribute significantly to an increase in image quality of the image based on the subject information. Typically, a high-frequency component of a photoacoustic wave tends to attenuate to a greater extent than does a low-frequency component of the photoacoustic wave, and thus the high-frequency component is less likely to contribute to an increase in image quality of the image based on the subject information than the low-frequency component. Then, storing a reception signal that does not contribute significantly to an increase in image quality in a memory as well leads to an increase in the used memory space.

In the meantime, of the photoacoustic waves generated inside the subject, a frequency component that does not attenuate to a great extent while propagating through the inside of the subject contributes significantly to an increase in image quality, and thus the significance of saving such a frequency component in a memory is high. Typically, a low-frequency component of a photoacoustic wave attenuates to a lesser extent than does a high-frequency component of the photoacoustic wave, and thus the low-frequency component contributes more to an increase in image quality of an image based on the subject information than does the high-frequency component.

Accordingly, the exemplary embodiments are generally directed to providing a photoacoustic apparatus that can selectively reduce the amount of data associated with a reception signal of a photoacoustic wave of a frequency component that does not contribute significantly to an increase in image quality.

It is to be noted that the reception signal as used in the present specification is an electric signal that is outputted from a transducer having received a photoacoustic wave and that has not been stored in a final-stage memory of a signal data acquisition unit. In addition, reception-signal data as used in the present specification is signal data that is stored in the final-stage memory of the signal data acquisition unit.

A photoacoustic wave generated as pulsed light emitted from a light source travels from the surface of a subject to a portion deep inside the subject propagates through the inside of the subject and then reaches an acoustic wave receiving element. The photoacoustic wave generated inside the subject propagates through the inside of the subject while being subjected to an influence of frequency-dependent attenuation (FDA). For example, the FDA of a normal breast is approximately 0.75 dB/cm/MHz, and as the frequency of a photoacoustic wave is higher, the photoacoustic wave attenuates to a greater extent while propagating through a living body. Meanwhile, the FDA of an acoustic matching material composed of water, gel, or the like is small enough to be ignored as compared with the FDA of a living body, and thus in the description of the present exemplary embodiment, attenuation of an acoustic wave occurring inside the acoustic matching material is ignored.

Therefore, typically, as the distance a photoacoustic wave propagates through the inside of the subject increases, a high-frequency component of the photoacoustic wave attenuates to a greater extent inside the subject than does a low-frequency component, due to an influence of the attenuation of the photoacoustic wave. In other words, as the distance the photoacoustic wave propagates through the inside of the subject increases, a low-frequency component becomes dominant in the frequency band characteristics of the photoacoustic wave received by the acoustic wave receiving element. Then, a reception signal of a high-frequency component whose signal intensity has decreased as the photoacoustic wave attenuates becomes a reception signal that does not contribute significantly to an increase in image quality of an image of the inside of the subject. Therefore, in such a case, the image quality of the image of the inside of the subject is less likely to decrease even when the image is formed without using a reception signal corresponding to the high-frequency component of the photoacoustic wave.

Accordingly, in the present exemplary embodiment, a sampling frequency is set at which an acoustic wave of a low-frequency component, which is contained dominantly in the acoustic wave, can be selectively sampled. Thus, the amount of data associated with a reception signal corresponding to an acoustic wave of a high-frequency component can be reduced.

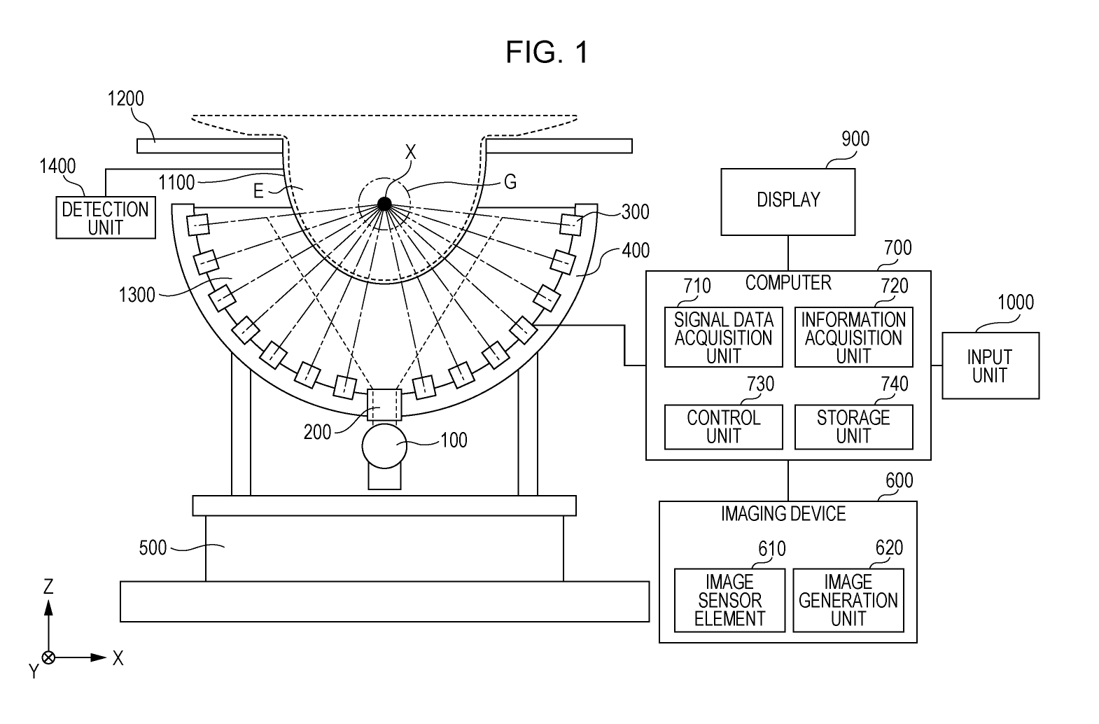

Hereinafter, a photoacoustic apparatus according to the present exemplary embodiment will be described. FIG. 1 is a schematic diagram illustrating the photoacoustic apparatus according to the present exemplary embodiment.

The photoacoustic apparatus illustrated in FIG. 1 acquires information on a subject E (subject information) on the basis of a reception signal of a photoacoustic wave generated through a photoacoustic effect.

Examples of the subject information that can be acquired with the photoacoustic apparatus according to the present exemplary embodiment include an initial sound pressure distribution of a photoacoustic wave, an optical energy absorption density distribution, an absorption coefficient distribution, and a concentration distribution of a substance forming a subject. Examples of the concentration of a substance include oxygen saturation, oxyhemoglobin concentration, deoxyhemoglobin concentration, and total hemoglobin concentration. The total hemoglobin concentration is the sum of the oxyhemoglobin concentration and the deoxyhemoglobin concentration.

### Basic Configuration

The photoacoustic apparatus according to the present exemplary embodiment includes a light source 100, an optical system 200, a plurality of acoustic wave receiving elements 300, a support member 400, and a scanner 500 serving as a moving unit. The photoacoustic apparatus according to the present exemplary embodiment further includes an imaging device 600, a computer 700, a display 900 serving as a display unit, an input unit 1000, and a shape retaining unit 1100. The computer 700 includes a signal data acquisition unit 710, an information acquisition unit 720, a control unit 730, and a storage unit 740.

Hereinafter, each component of the photoacoustic apparatus and components used in the measurement will be described.

### Subject

The subject E is a target to be measured. Specific examples of the subject E include a living body, such as a breast, and, when the photoacoustic apparatus is to be adjusted, a phantom simulating acoustic characteristics and optical characteristic of a living body. The acoustic characteristics, specifically, are the propagation speed and the attenuation rate of acoustic waves; and the optical characteristic, specifically, are the absorption coefficient and the scattering coefficient of light. Examples of optical absorbers inside a living body serving as a subject include hemoglobin, water, melanin, collagen, and lipid. When a phantom is used, a substance that simulates the optical characteristic is injected into the phantom to serve as an optical absorber. For convenience, the subject E is indicated by a dotted line in FIG. 1.

### Light Source

The light source 100 emits pulsed light. To achieve a high-power light source, it is desirable to use a laser, but a light-emitting diode or the like may also be used. In order to effectively generate a photoacoustic wave, a subject needs to be irradiated with light in a sufficiently short period of time in accordance with the thermal properties of the subject. When the subject is a living body, it is desirable that the pulse duration of the pulsed light emitted from the light source 100 be no greater than several tens of nanoseconds. In addition, it is desirable that the wavelength of the pulsed light be approximately from 700 nm to 1200 nm, which is a near-infrared band called a window of the living body. Light in this band can reach a portion that is relatively deep inside the living body, and information on a portion deep inside the living body can thus be acquired. When the measurement is to be made only on the surface of the living body, visible light at a wavelength in a range approximately from 500 nm to 700 nm or light in the near-infrared band may be used. Furthermore, it is desirable that the pulsed light have a wavelength at which the measurement target has a high absorption coefficient for the pulsed light.

### Optical System

The optical system 200 guides the pulsed light emitted from the light source 100 to the subject E. Specifically, the optical system 200 is an optical device, such as a lens, a mirror, a prism, an optical fiber, and a diffusion plate. When the light is guided, the shape or the optical density of the light may be changed by such an optical device so that the light has a desired light distribution. The optical device is not limited to those mentioned above, and any optical device that achieves a similar function may be used. The optical system 200 according to the present exemplary embodiment is configured to illuminate a region at the center of curvature of a hemisphere.

In addition, with regard to the intensity of light that is permitted to irradiate a biological tissue, the maximum permissible exposure (MPE) is defined by the safety standards such as those indicated below (IEC 60825-1: Safety of laser products, JIS C 6802: Safety of laser products, FDA: 21CFR Part 1040.10, ANSI 2136.1: Laser Safety Standards, etc.). The MPE is defined in terms of the intensity of light that is permitted to irradiate per unit area. Thus, as a broader area on the surface of the subject E is irradiated at once with light, a larger amount of light can be guided to the subject E; therefore, a photoacoustic wave can be received at a high signal-to-noise (S/N) ratio. Accordingly, it is preferable to increase the profile to a certain extent by converging the light with a lens, as indicated by a broken line illustrated in FIG. 1.

### Acoustic Wave Receiving Element

The acoustic wave receiving elements 300 receive photoacoustic waves and convert the photoacoustic waves to electric signals. It is desirable that the acoustic wave receiving elements 300 have high receiving sensitivity to the photoacoustic waves from the subject E and have a broad frequency band.

The acoustic wave receiving elements 300 can be formed of a piezoelectric ceramic material as exemplified by lead zirconate titanate (PZT), a piezoelectric polymer film material as exemplified by polyvinylidene fluoride (PVDF), or the like. Alternatively, element that are not piezoelectric elements may be used. For example, electrostatic capacitance elements, such as capacitive micro-machined ultrasonic transducers (CMUTs), or acoustic wave receiving elements that are constituted by Fabry-Perot interferometers can be used.

Typically, the receiving sensitivity characteristics of an acoustic wave receiving element show the highest sensitivity to an acoustic wave that is incident normally on a receiving surface, and the receiving sensitivity decreases as the angle of incidence increases. In the present exemplary embodiment, when the maximum value of the receiving sensitivity is represented by S and the angle of incidence at which the receiving sensitivity is S/2 or one-half the maximum value is represented by a, a range in which a photoacoustic wave is incident on the receiving surface of an acoustic wave receiving element 300 at an angle that is no greater than a is defined as a receiving range in which the acoustic wave receiving element 300 can receive the photoacoustic wave at high sensitivity. In FIG. 1, the direction in which each of the acoustic wave receiving elements 300 shows the highest receiving sensitivity is indicated by a dashed-dotted line. Hereinafter, an axis that extends in the direction in which the receiving sensitivity is highest is also referred to as a directional axis in the present specification.

### Support Member

The support member 400 is a substantially hemispherical receptacle and supports the plurality of acoustic wave receiving elements 300 at a hemispherical inner surface thereof. In addition, the optical system 200 is disposed at a base portion (pole) of the hemispherical support member 400. The inner space of the hemisphere is filled with an acoustic matching material 1300, which will be described later. In the present exemplary embodiment, the plurality of acoustic wave receiving elements 300 are disposed so as to follow the hemispherical shape, as illustrated in FIG. 1. A point X indicates the center of curvature of the hemispherical support member 400. The support member 400 supports the plurality of acoustic wave receiving elements 300 such that the directional axes of the plurality of acoustic wave receiving elements 300 converge.

As the directional axes of the plurality of acoustic wave receiving elements 300 converge at the center point X of curvature of the hemispherical shape or the vicinity thereof, a region G in which high-accuracy visualization is possible is formed with its center being located at the center point X of curvature. In the present specification, the region G in which high-accuracy visualization is possible is referred to as a high-sensitivity region. As the support member 400 is moved by the scanner 500, which will be described later, relative to the subject E, the high-sensitivity region G is moved, and the subject information in a broad range can be visualized with high accuracy.

The high-sensitivity region G can be considered as a substantially spherical region having a radius r, expressed in Expression (1), and with its center being located at the center point X of curvature at which the highest resolution R_{H} can be obtained.

$$r = \frac{{r}_{0}}{{φ}_{d}} \sqrt{{R}^{2} - {R}_{H}^{2}} \qquad ( 1 )$$

In Expression (1), R is the lower limit resolution in the high-sensitivity region G; R_{H} is the highest resolution; r₀ is the radius of the hemispherical support member 400; and φ_{d} is the diameter of the acoustic wave receiving element 300. R, for example, may be set to a resolution that is one-half the highest resolution that is obtained at the center point X of curvature, as described above.

When the high-sensitivity region G is defined as a substantially spherical region with its center being located at the center point X of curvature of the probe, the range of the high-sensitivity region G at each position on a two-dimensional scan of the probe can be estimated through Expression (1) on the basis of the shape of the high-sensitivity region G and the position of the probe (i.e., the center point X of curvature).

It is to be noted that the arrangement of the plurality of acoustic wave receiving elements 300 is not limited to the hemispherical shape as illustrated in FIG. 1. The plurality of acoustic wave receiving elements 300 may be arranged in any manner as long as the directional axes of the plurality of acoustic wave receiving elements 300 converge and a predetermined high-sensitivity region can be formed. In other words, it is sufficient if the plurality of acoustic wave receiving elements 300 are arranged along a curved shape forming a predetermined region so that a predetermined high-sensitivity region G is formed. A curved surface as used in the present specification includes a true spherical shape and a spherical surface that includes an opening of a hemispherical shape or the like. In addition, a curved surface includes a surface that has concavities and convexities therein to an extent that allows the surface to be considered as being spherical, an ellipsoidal surface (a shape obtained by extending an ellipse into a three-dimensional shape, and the surface thereof is quadric) that can be considered as being spherical.

When a plurality of acoustic wave receiving elements are disposed so as to follow a support member having a shape obtained by sectioning a sphere along a given plane, the directional axes most converge at the center of curvature of the shape of the support member. The hemispherical support member 400 described in the present exemplary embodiment is an example of such a support member having a shape obtained by sectioning a sphere along a given plane. In the present specification, such a shape obtained by sectioning a sphere along a given plane is referred to as a sphere-based shape. A plurality of acoustic wave receiving elements that are supported by a support member having such a sphere-based shape are supported on a spherical surface.

The optical system 200 serving as irradiation optics for guiding the light is disposed on the base of the support member 400.

It is to be noted that as long as a desired high-sensitivity region can be formed, the directional axes of the respective acoustic wave receiving elements do not necessarily have to meet. In addition, it is sufficient if the directional axes of at least some of the plurality of acoustic wave receiving elements 300 supported by the support member 400 converge at a specific region so that a photoacoustic wave generated in the specific region can be received with high sensitivity. In other words, it is sufficient if the plurality of acoustic wave receiving elements 300 are disposed on the support member 400 such that at least some of the plurality of acoustic wave receiving elements 300 can receive a photoacoustic wave generated in a high-sensitivity region with high sensitivity.

It is preferable that the support member 400 be formed of a metal material or the like having a large mechanical strength.

Scanner

The scanner 500 moves the position of the support member 400 in X-, Y-, and Z-directions indicated in FIG. 1 so as to change the position of the support member 400 relative to the subject E. Thus, the scanner 500 includes X-, Y-, and Z-direction guide mechanisms (not illustrated), X-, Y-, and Z-direction driving mechanisms (not illustrated), and a position sensor (not illustrated) that receives the position of the support member 400 in the X-, Y-, and Z-directions. As illustrated in FIG. 1, the support member 400 is placed on the scanner 500; therefore, it is preferable that the guide mechanisms be constituted by linear guides that can stand a heavy load. The driving mechanisms can be constituted by lead screw mechanisms, link mechanisms, gear mechanisms, hydraulic mechanisms, or the like. A motor or the like may be used to produce driving force. The position sensor can be constituted by a potentiometer that includes an encoder, a variable resistor, or the like.

It is to be noted that, in the exemplary embodiments, it is sufficient if the positional relationship of the subject E and the support member 400 changes; thus, the support member 400 may be fixed, and the subject E may be moved. If the subject E is to be moved, a configuration that moves the subject E by moving a support unit (not illustrated) that supports the subject E may be considered. Alternatively, the subject E and the support member 400 may both be moved.

The scanner 500 is not limited to a scanner that changes the positional relationship of the subject E and the support member 400 three-dimensionally but may change the stated positional relationship one-dimensionally or two-dimensionally.

Although it is desirable that the subject E and/or the support member 400 be moved continuously, the subject E and/or the support member 400 may be moved stepwise. It is desirable that the scanner 500 be an electromotive stage, but the scanner 500 may also be a manually operated stage. The configuration of the scanner 500 is not limited to the examples described above, and the scanner 500 may have any configuration that enables at least one of the subject E and the support member 400 to be moved.

### Imaging Device

The imaging device 600 generates image data of the subject E and outputs the generated image data to the computer 700. The imaging device 600 includes an image sensor element 610 and an image generation unit 620. The image generation unit 620 analyzes a signal outputted from the image sensor element 610 so as to generate image data of the subject E, and stores the generated image data in the storage unit 740 of the computer 700.

For example, the image sensor element 610 can be constituted by an optical image sensor element, such as a charge-coupled device (CCD) sensor and a complementary metal-oxide semiconductor (CMOS) sensor. Alternatively, the image sensor element 610 can be constituted by an acoustic image sensor element, such as a piezoelectric element and a CMUT, that transmits and receives an acoustic wave. Some of the plurality of acoustic wave receiving elements 300 may be used as the image sensor element 610. The image sensor element 610 may be constituted by any element as long as the image generation unit 620 can generate an image of the subject on the basis of a signal outputted from the image sensor element 610.

The image generation unit 620 is constituted by an element such as a central processing unit (CPU), a graphics processing unit (GPU), and an analog/digital (A/D) converter and a circuit such as a field programmable gate array (FPGA) and an application specific integrated circuit (ASIC). It is possible that the computer 700 also fulfills the function of the image generation unit 620. Specifically, an arithmetic unit of the computer 700 can be used as the image generation unit 620.

The imaging device 600 may be provided separately from the photoacoustic apparatus.

### Computer

The computer 700 includes the signal data acquisition unit 710, the information acquisition unit 720, the control unit 730, and the storage unit 740.

The signal data acquisition unit 710 converts time-series reception signals outputted from the plurality of acoustic wave receiving elements 300 to digital signals and stores the digital signals as reception-signal data.

The information acquisition unit 720 generates subject information on the basis of the reception-signal data stored by the signal data acquisition unit 710. The reception-signal data is time-series signal data, and the subject information is two-dimensional or three-dimensional spatial data. Two-dimensional spatial data may also be referred to as pixel data, and three-dimensional spatial data may also be referred to as voxel data or volume data.

For example, as an image reconstruction algorithm for acquiring subject information, time-domain or Fourier-domain back projection that is typically used in a tomography technique is used. If it is possible to spend an extended period of time on the reconstruction, an image reconstruction technique, such as an inverse problem analysis through iteration, may also be employed.

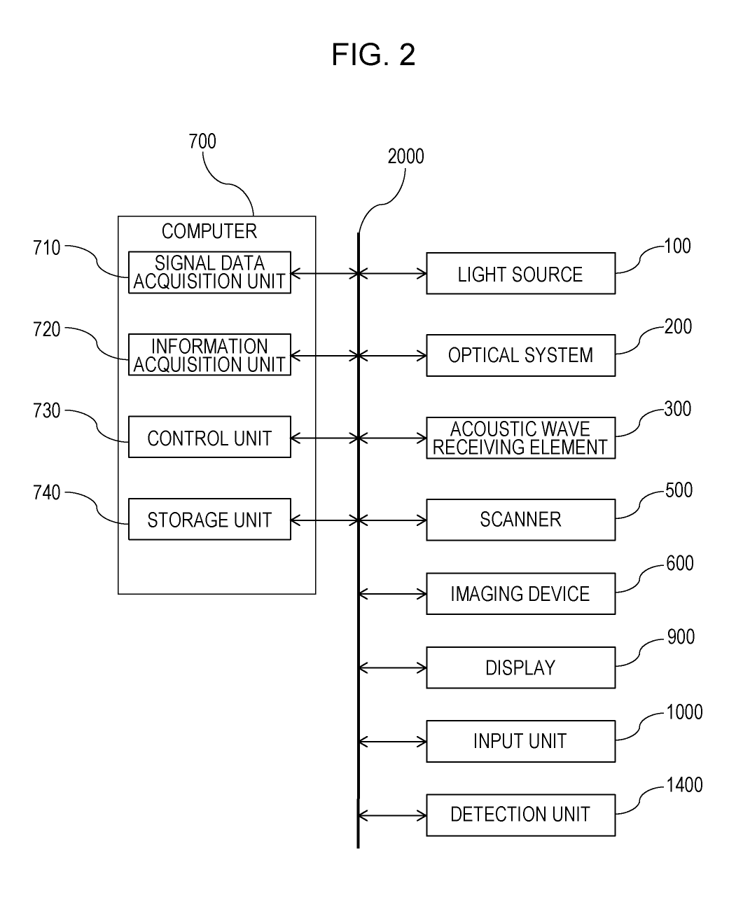

The control unit 730 can control the operations of the components constituting the photoacoustic apparatus through a bus 2000, as illustrated in FIG. 2. The control unit 730 is typically constituted by a CPU. As the control unit 730 reads out a program, stored in the storage unit 740, for controlling the operation, the operation of the photoacoustic apparatus is controlled. The storage unit 740, in which the program is stored, is a non-transitory recording medium.

Each of the signal data acquisition unit 710 and the information acquisition unit 720 includes an arithmetic unit and a storage unit. The arithmetic unit is constituted by an arithmetic element, such as a CPU, a GPU, and an A/D converter, and an arithmetic circuit, such as an FPGA and an ASIC. The arithmetic unit does not have to be constituted by a single element and a single circuit but may be constituted by a plurality of elements and a plurality of circuits. Each of the processes according to the exemplary embodiments may be executed by any element or circuit. The storage unit is constituted by a storage medium, such as a read-only memory (ROM), a random-access memory (RAM), and a hard disk. The storage unit does not have to be constituted by a single storage medium but may be constituted by a plurality of storage media.

Although the signal data acquisition unit 710, the information acquisition unit 720, the control unit 730, and the storage unit 740 are described as separate entities, for convenience, in the present specification, a common element may implement the function of each of the aforementioned units. For example, an arithmetic unit may carry out arithmetic processes implemented by the signal data acquisition unit 710, the information acquisition unit 720, and the control unit 730.

It is preferable that the computer 700 be capable of pipeline processing of a plurality of signals simultaneously. Through this configuration, the time it takes to acquire subject information can be reduced.

### Acoustic Matching Material

The acoustic matching material 1300 is used to fill a space between the subject E and the acoustic wave receiving elements 300 so as to acoustically couple the subject E and the acoustic wave receiving elements 300. In the present exemplary embodiment, a space between the shape retaining unit 1100 and the subject E is also filled with the acoustic matching material 1300.

A space between the acoustic wave receiving elements 300 and the shape retaining unit 1100 can also be filled with the acoustic matching material 1300. The space between the acoustic wave receiving elements 300 and the shape retaining unit 1100 and the space between the shape retaining unit 1100 and the subject E may be filled with different acoustic matching materials.

It is preferable that the acoustic matching material 1300 be a material that is less likely to cause a photoacoustic wave traveling therethrough to attenuate. It is preferable that the acoustic matching material 1300 be a material whose acoustic impedance is close to the acoustic impedances of the subject E and of the acoustic wave receiving elements 300. In addition, it is even preferable that the acoustic matching material 1300 be a material whose acoustic impedance lies between the acoustic impedance of the subject E and the acoustic impedance of the acoustic wave receiving elements 300. Furthermore, it is preferable that the acoustic matching material 1300 be a material that transmits pulsed light emitted from the light source 100. In addition, it is preferable that the acoustic matching material 1300 be a liquid. Specifically, water, castor oil, gel, or the like can be used as the acoustic matching material 1300.

The acoustic matching material 1300 may be provided separately from the photoacoustic apparatus according to the exemplary embodiments.

### Display

The display 900 displays subject information outputted from the computer 700 in the form of a distribution image or numeric data. Typically, a liquid crystal display or the like is used, but a display of a different system, such as a plasma display, an organic electroluminescence (EL) display, and a field emission display (FED), may also be used. The display 900 may be provided separately from the photoacoustic apparatus according to the exemplary embodiments.

### Input Unit

The input unit 1000 is configured to allow a user to specify desired information in order to input desired information into the computer 700. The input unit 1000 can be constituted by a keyboard, a mouse, a touch panel, a dial, a button, or the like. When the input unit 1000 is to be constituted by a touch panel, the display 900 may be constituted by a touch panel that is to be used as the input unit 1000 as well. The input unit 1000 may be provided separately from the photoacoustic apparatus according to the exemplary embodiments.

### Shape Retaining Unit

The shape retaining unit 1100 is a member for retaining the shape of the subject E constant. The shape retaining unit 1100 is mounted to a mounting unit 1200. In a case in which multiple shape retaining units are to be used to retain the shapes of respective subjects E, it is preferable that the mounting unit 1200 be configured such that the multiple shape retaining units can be mounted to the mounting unit 1200.

When the subject E is to be irradiated with light through the shape retaining unit 1100, it is preferable that the shape retaining unit 1100 be transparent to the irradiation light. For example, the shape retaining unit 1100 can be formed of polymethylpentene, polyethylene terephthalate, or the like.

In a case in which the subject E is a breast, it is preferable that the shape retaining unit 1100 has a shape obtained by sectioning a sphere along a given plane, in order to retain the shape of the breast constant with little deformation. The shape of the shape retaining unit 1100 can be designed as appropriate in accordance with the cubic content of the subject or a desired shape to be obtained when the subject is held by the shape retaining unit 1100. It is preferable that the shape retaining unit 1100 be configured such that the shape retaining unit 1100 fits the external shape of the subject E and the shape of the subject E becomes substantially the same as the shape of the shape retaining unit 1100. It is to be noted that the photoacoustic apparatus may carry out the measurement without the shape retaining unit 1100.

### Example of Method for Determining Sampling Frequency

Subsequently, an example of a method for determining a sampling frequency for selectively storing a reception signal of a frequency component that can be received at high intensity in the present exemplary embodiment will be described.

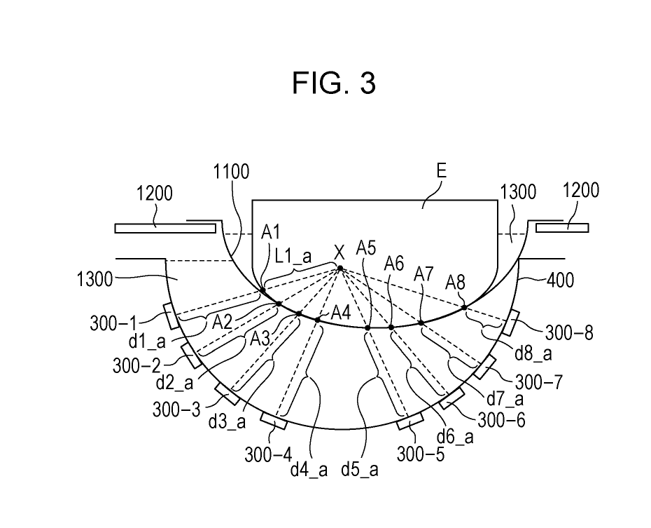

When the plurality of acoustic wave receiving elements 300 disposed as illustrated in FIG. 3 are to be used, a photoacoustic wave generated at the center X (the center point of the high-sensitivity region) of curvature of the support member at which the directionalities of the acoustic wave receiving elements 300 converge can be received with high sensitivity. Meanwhile, the distance from the surface of the subject to the center X of curvature as viewed from the plurality of acoustic wave receiving elements 300 toward the center X of curvature differs. In this case, a distance LN_a (N=1 to 8) from the surface of the subject to the center X of curvature as viewed from an acoustic wave receiving element 300-N(N=1 to 8) corresponds to the length of a line segment connecting a point AN (N=1 to 8) and the center X of curvature. For example, the distance L1_{—}a from the surface of the subject to the center X of curvature as viewed from an acoustic wave receiving element 300-1 corresponds to the length of a line segment connecting a point A1 and the center X of curvature. For example, in the case of the example illustrated in FIG. 3, the distance LN_a (N=1 to 8) from the surface of the subject to the center X of curvature as viewed from the acoustic wave receiving element 300-N(N=1 to 8) increases as N changes from N=1 to N=8. In this case, a photoacoustic wave generated at the center X of curvature and reaching the acoustic wave receiving element 300-N having a larger value of N attenuates to a greater extent. In particular, a high-frequency component contained in a photoacoustic wave reaching an acoustic wave receiving element 300-N having a larger value of N attenuates to a greater extent than a high-frequency component contained in a photoacoustic wave reaching an acoustic wave receiving element 300-N having a smaller value of N.

Accordingly, in the present exemplary embodiment, the sampling frequency is varied between an acoustic wave receiving element that receives a photoacoustic wave in which a high-frequency component attenuates to a great extent and a low-frequency component is dominant and an acoustic wave receiving element that receives a photoacoustic wave in which a high-frequency component does not attenuate to a great extent. For example, the sampling frequency of an acoustic wave receiving element 300-8 that receives a photoacoustic wave in which a high-frequency component attenuates to a great extent is set to be lower than the sampling frequency of the acoustic wave receiving element 300-1 that receives a photoacoustic wave in which a high-frequency component does not attenuate to a great extent. In the acoustic wave receiving element 300-8, as the sampling frequency is reduced, a photoacoustic wave of a high-frequency component is not sampled with a high degree of fidelity, and a photoacoustic wave of a low-frequency component is selectively sampled. Meanwhile, as the sampling frequency of the acoustic wave receiving element 300-8 is reduced, the amount of data associated with the reception-signal data corresponding to the acoustic wave receiving element 300-8 becomes smaller than the amount of data associated with the reception-signal data corresponding to the acoustic wave receiving element 300-1. However, the photoacoustic wave of a high-frequency component reaching the acoustic wave receiving element 300-8 has attenuated and the signal intensity thereof is being reduced, and such a high-frequency component thus results in data that does not contribute significantly to an increase in image quality of an image of the inside of the subject E. Therefore, even if such a photoacoustic wave cannot be sampled with a high degree of fidelity, the image quality of the image of the inside the subject is less likely to be reduced.

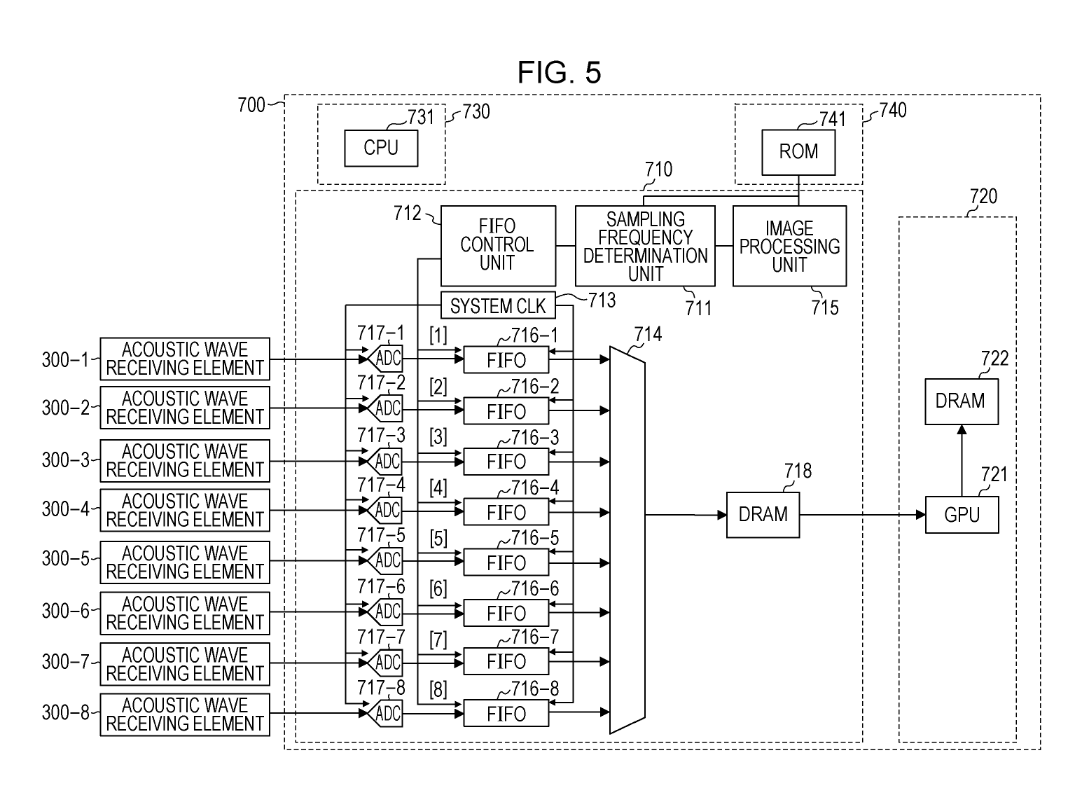

Accordingly, a sampling frequency determination unit 711 illustrated in FIG. 5 sets the sampling frequencies as described above on the basis of information that is based on measurement positions, and thus a frequency component that reaches an acoustic wave receiving element at high intensity can selectively be stored.

An attenuation ΔI [dB] of a photoacoustic wave having a frequency f [MHz] occurring when the photoacoustic wave propagates through the inside of a subject having the FDA of a [dB/cm/MHz] to a depth L [cm] is expressed through Expression (2).

ΔI=α·L·f (2)

In Expression (2), when a permissible attenuation by which a sound pressure of a photoacoustic wave held when the photoacoustic wave is generated falls below an S/N ratio at which the photoacoustic wave contributes significantly to an increase in image quality is represented by ΔI′, a reception signal of a photoacoustic wave at a frequency that is higher than the frequency f indicated in Expression (3) may become a frequency component that does not contribute significantly to an increase in image quality.

$$f = \frac{Δ {I}^{′}}{α \cdot L} \qquad ( 3 )$$

Accordingly, the sampling frequency determination unit 711 samples time-series reception signals at a sampling frequency that allows the frequency f determined through Expression (3) to be sufficiently sampled, and thus a frequency component that is no greater than the frequency f can sufficiently be sampled. Specifically, the sampling frequency determination unit 711 determines a sampling frequency that allows, among frequency components of a photoacoustic wave generated at a specific position, a frequency component whose attenuation is no greater than the permissible attenuation to be sampled. In addition, the sampling frequency determination unit 711 determines a sampling frequency that does not allow, among frequency components of a photoacoustic wave generated at a specific position, a frequency component whose attenuation is greater than the permissible attenuation to be sampled. Through this configuration, a frequency component that contributes significantly to an increase in image quality is sampled at a sufficient sampling frequency, and a frequency component that does not contribute significantly to an increase in image quality is not sampled with a high degree of fidelity; thus, the amount of data can be reduced.

For example, it is preferable that ΔI′ be set so as to result in an S/N ratio that does not contribute significantly to an increase in image quality, in a case in which the sound pressure of a photoacoustic wave held when the photoacoustic wave is generated attenuates by no less than 10 dB. When ΔI′ is set to a small value, a frequency component that contributes significantly to an increase in image quality may become unable to be sampled with a high degree of fidelity; therefore, it is preferable that ΔI′ be set to no less than 5 dB. In other words, it is preferable that ΔI′ be set to no less than 5 dB and no greater than 10 dB. In addition, ΔI′ can be set as appropriate in accordance with the minimum receiving sound pressure of an acoustic wave receiving element. The user can input the value of ΔI′ through the input unit 1000 so as to set ΔI′.

The FDA can be set as appropriate through the input unit 1000 in accordance with the type of the subject. Alternatively, if the type of the subject is known in advance, the value of the FDA can be stored in advance in a ROM 741 serving as the storage unit 740.

The sampling frequency may be determined with distance-dependent attenuation caused by energy dissipation due to spherical wave propagation, cylindrical wave propagation, and so on in the attenuation of acoustic waves taken into consideration.

It is preferable that the sampling frequency be set such that a frequency determined through Expression (3) in accordance with the sampling theorem can sufficiently be sampled. For example, typically, it is preferable that the sampling frequency be set to a frequency that is no less than twice the frequency f determined through Expression (3) in accordance with the sampling theorem.

However, as the sampling frequency increases, the amount of data associated with the reception-signal data increases as well; therefore, it is not preferable to increase the sampling frequency unlimitedly. Accordingly, the present inventor has conducted diligent investigation and found that when the sampling frequency is set to a frequency that is no less than ten times the frequency f, obtained data does not contribute significantly to data reproducibility in a photoacoustic apparatus. In addition, it was found that a component of the frequency f can be sufficiently sampled at a sampling frequency that is approximately four times the frequency f. Accordingly, it is preferable that the sampling frequency be set to a frequency that is no greater than ten times the frequency f. In addition, in order to reduce the amount of data associated with the reception signals, it is preferable that the sampling frequency be set to a frequency that is no greater than four times the frequency f.

In other words, it is preferable that the sampling frequency be set to a frequency that is no less than twice the frequency f and no greater than ten times the frequency f. Furthermore, in order to reduce the amount of data associated with the reception signals, it is preferable that the sampling frequency be set to a frequency that is no less than twice the frequency f and no greater than four times the frequency f.

As the sampling frequency of each acoustic wave receiving element is set in the manner described above, data of a reception signal of a frequency component reaching each acoustic wave receiving element at a high intensity can selectively be acquired. Meanwhile, the amount of data associated with a reception signal of a frequency component whose intensity has been reduced as being attenuated can be reduced. In this manner, the sampling frequency of each acoustic wave receiving element can be set individually in accordance with a frequency component of a photoacoustic wave reaching each acoustic wave receiving element.

### Operation of Photoacoustic Apparatus

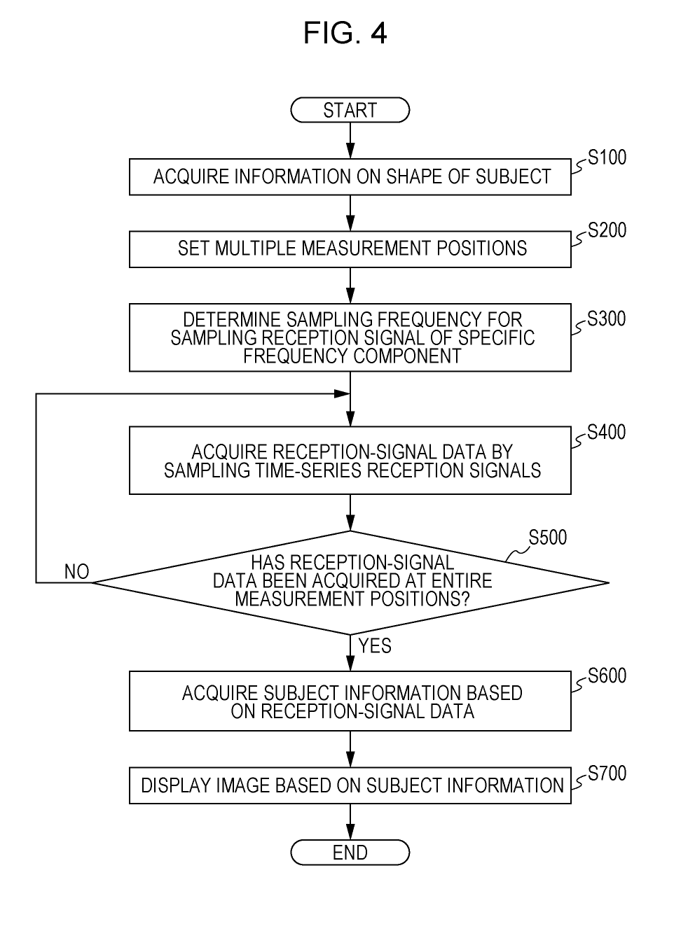

Subsequently, with reference to the flowchart illustrated in FIG. 4, a method for storing, in a memory, data of a photoacoustic wave generated inside a subject selectively on the basis of the shape information of the subject will be described.

### S100: Process of Acquiring Shape Information of Subject

First, the subject E is placed on the shape retaining unit 1100, and the space between the support member 400 and the shape retaining unit 1100 and the space between the shape retaining unit 1100 and the subject E are filled with the acoustic matching material 1300.

Subsequently, the sampling frequency determination unit 711 of the signal data acquisition unit 710 acquires information that is based on the shape of the subject E. The information that is based on the shape of the subject as used herein is information on the position coordinate on the surface of the subject E or information on the type of the shape retaining unit 1100. In addition, acquiring the information that is based on the shape of the subject E means that the sampling frequency determination unit 711 receives information that is based on the shape of the subject.

Hereinafter, a method through which the sampling frequency determination unit 711 acquires the information that is based on the shape of the subject will be described.

An image processing unit 715 first reads out, from the ROM 741, image data of the subject E acquired by the imaging device 600. Subsequently, the image processing unit 715 calculates the coordinate information on the surface of the subject E on the basis of the image data of the subject E, and outputs the calculated coordinate information to the sampling frequency determination unit 711. For example, the image processing unit 715 may calculate the coordinate information on the surface of the subject E by using a three dimensional measurement technique, such as a stereo method, on the basis of a plurality of pieces of image data. Then, the sampling frequency determination unit 711 can receive the information on the position coordinate on the surface of the subject E outputted from the image processing unit 715 and thus acquire the shape information of the subject.

Alternatively, information on the position coordinate on the surface of the shape retaining unit 1100 that is known in advance can be stored in the ROM 741. Then, the sampling frequency determination unit 711 can read out the information on the position coordinate on the surface of the shape retaining unit 1100 from the ROM 741 and thus acquire the information on the position coordinate on the surface of the subject E.

As another alternative, a detection unit 1400 can be provided that detects the type of the shape retaining unit mounted to the mounting unit 1200 and outputs information on the type of the shape retaining unit to the computer 700. Then, the sampling frequency determination unit 711 can receive the information on the type of the shape retaining unit outputted from the detection unit 1400 and thus acquire the information that is based on the shape of the subject. For example, the detection unit 1400 can be constituted by a reader that reads an ID chip, provided on the shape retaining unit, that indicates the type of the shape retaining unit. Through this configuration, the information that is based on the shape of the subject can be acquired without a calculation.

As yet another alternative, the user inputs the type of the shape retaining unit to be used through the input unit 1000, and the input unit 1000 outputs the inputted information to the sampling frequency determination unit 711. Then, the sampling frequency determination unit 711 can receive the information on the type of the shape retaining unit outputted from the input unit 1000 and thus acquire the information that is based on the shape of the subject. Through this configuration, the information that is based on the shape of the subject can be acquired without a calculation.

When it is assumed that the type of the shape retaining unit does not change and that the dimensions of the shape retaining unit do not change according to the specification of the apparatus, the information that is based on the shape of the subject and is used by the sampling frequency determination unit 711 may be held constant.

In a case in which the photoacoustic apparatus carries out the measurement multiple times, information that is based on the shape of the subject acquired through this process may be used in a subsequent instance of the measurement. In addition, in a case in which the photoacoustic apparatus carries out the measurement multiple times, this process can be carried out at any desired timing. For example, the process may be carried out at each instance of the measurement, or the process may be carried out every several instances of the measurement.

When the process is carried out at each instance of the measurement, even if the shape of the subject changes between measurements, a subsequent process can be carried out each time on the basis of the accurate information that is based on the shape of the subject.

In a case in which the information that is based on the shape of the subject is not used in processes described later, this process does not need to be carried out.

### S200: Process of Setting Plurality of Measurement Positions

Subsequently, a CPU 731 serving as the control unit 730 sets a plurality of measurement positions and stores information on the plurality of set measurement positions in the ROM 741. In the process of S400 described later, the subject E is irradiated with the light when the support member 400 is located at the plurality of set measurement positions. In other words, the information on the plurality of measurement positions corresponds to the information on the positions of the support member 400 at a plurality of light irradiation timings. Hereinafter, the measurement position refers to the position of the support member 400 at the time of light irradiation.

It is preferable that the CPU 731 set the plurality of measurement positions such that the subject E is irradiated with the light when the high-sensitivity region G is formed inside the subject E. Accordingly, the CPU 731 can set the plurality of measurement positions such that the subject E is irradiated with the light when the high-sensitivity region G is formed inside the subject E on the basis of the shape information of the subject E acquired in S100. The position and the dimensions of the high-sensitivity region G can be calculated in advance from the arrangement of the plurality of acoustic wave receiving elements 300 on the support member 400 and can be stored in the ROM 741. Therefore, the CPU 731 can set the plurality of measurement positions on the basis of the information on the position coordinate on the surface of the subject E and the position and the dimensions of the high-sensitivity region G stored in the ROM 741. In particular, the CPU 731 can set the plurality of measurement positions such that the subject E is irradiated with the light when the high-sensitivity region G is formed inside the subject E on the basis of the aforementioned pieces of information.

In addition, it is preferable that the CPU 731 set the plurality of measurement positions such that the center of the high-sensitivity region G is located inside the subject E. In the case of the present exemplary embodiment, it is preferable that a movement region be set such that the center of curvature of the hemispherical support member 400 is located inside the subject E at each measurement position. Furthermore, it is even preferable that the CPU 731 set the plurality of measurement positions such that the center of the high-sensitivity region G corresponding to an outermost periphery of the movement region follows along the outer edge of the subject E.

The CPU 731 can set the plurality of measurement positions such that the positions of the support member 400 are evenly spaced among the light irradiation timings.

The user may input the plurality of measurement positions through the input unit 1000, and the CPU 731 may set the plurality of measurement positions on the basis of the information outputted from the input unit 1000.

As the plurality of measurement positions are set as described above, although the movement region of the support member is small, photoacoustic waves generated in a broad range in the subject E can be received with high sensitivity. As a result, the subject information of the inside of the subject E to be acquired has a high resolution in a broad range.

In addition, the CPU 731 serving as a path setting unit can set, as appropriate, a moving path of the support member 400 that passes through the plurality of measurement positions set within the movement region. For example, the CPU 731 can move the support member 400 along a moving path that is close to a circular motion. As such a moving path is used, a change in the acceleration of the support member 400 in the direction in which the support member 400 moves is small; thus, a vibration of the acoustic matching material 1300 or a vibration of the photoacoustic apparatus can be suppressed. Here, a moving path that is close to a circular motion refers to a moving path that bends at an angle less than 90° relative to the traveling direction.

The user may input the moving path through the input unit 1000, and the CPU 731 may set the moving path on the basis of the information outputted from the input unit 1000.

### S300: Process of Determining Sampling Frequency for Sampling Reception Signal of Specific Frequency Component

Subsequently, the signal data acquisition unit 710 determines the sampling frequency that allows each of the plurality of acoustic wave receiving elements 300 to selectively acquire, with the method described above, data associated with a reception signal of a frequency component that reaches the acoustic wave receiving element 300 at a high intensity.

Hereinafter, with reference to FIGS. 3 and 5, a specific example of the method for determining the sampling frequency will be described. FIG. 5 illustrates a specific example of the configuration of the computer 700.

The sampling frequency determination unit 711 acquires information on the position coordinates of the plurality of acoustic wave receiving elements 300 and the position coordinate of the center X of curvature on the basis of the information on the measurement positions acquired in S200. Typically, the arrangement of the plurality of acoustic wave receiving elements 300 is known in advance; thus, the position coordinates of the plurality of acoustic wave receiving elements 300 corresponding to the respective positions of the support member 400 and the position coordinate of the center X of curvature can be calculated in advance, and the calculated position coordinates can be stored in the ROM 741. Then, the sampling frequency determination unit 711 can read out from the ROM 741 and acquire the position coordinates of the plurality of acoustic wave receiving elements 300 corresponding to the respective measurement positions and the position coordinate of the center X of curvature on the basis of the information on the measurement positions acquired in S200. Alternatively, the sampling frequency determination unit 711 may calculate the position coordinates of the plurality of acoustic wave receiving elements 300 corresponding to the respective positions of the support member 400 and the position coordinate of the center X of curvature on the basis of the information on the measurement positions acquired in S200 and the information on the arrangement of the plurality of the acoustic wave receiving elements 300.

Subsequently, the sampling frequency determination unit 711 calculates the distances L1_{—}a through L8_{—}a on the basis of the position coordinates of the plurality of acoustic wave receiving elements 300, the position coordinate of the center X of curvature, and the position coordinate on the surface of the subject E acquired in S100.

The sampling frequency determination unit 711 then obtains the sampling frequencies corresponding to the plurality of acoustic wave receiving elements 300 through Expression (3) on the basis of the information on the distances L1_{—}a through L8_{—}a.

Sampling frequencies for the plurality of acoustic wave receiving elements 300 that correspond to subjects of any shapes and any measurement positions can be calculated, and the calculated sampling frequencies can be stored in the ROM 741. Then, the sampling frequency determination unit 711 can read out from the ROM 741 and acquire a sampling frequency corresponding to a given shape of a subject and a given measurement position on the basis of the information that is based on the shape of the subject and the information on the measurement positions.

In a case in which the shape retaining unit 1100 is replaceable, sampling frequencies for the plurality of acoustic wave receiving elements 300 that correspond to various types of the shape retaining unit 1100 and the respective measurement positions can be calculated in advance, and the calculated sampling frequencies can be stored in the ROM 741. Then, the sampling frequency determination unit 711 can read out from the ROM 741 and acquire a sampling frequency corresponding to the plurality of acoustic wave receiving elements 300 on the basis of the information on the type of the shape retaining unit 1100 and the information on the measurement positions.

In this manner, in the present exemplary embodiment, the sampling frequencies are determined that selectively reduce the amount of data associated with the reception-signal data corresponding to an attenuated component among components contained in a photoacoustic wave generated at the center of curvature of the support member 400 with the center of curvature serving as a reference. It is to be noted that, in this process, the sampling frequencies can also be set that selectively reduce the amount of data associated with the reception-signal data corresponding to an attenuated component among components contained in a photoacoustic wave generated at any given position aside from the center of curvature of the support member 400. For example, the sampling frequencies may be determined on the basis of a specific position within a target region to be imaged that is set by the user through the input unit 1000. In addition, the sampling frequencies may be determined on the basis of a position that is farthest from the probe within the set target region serving as the specific position. Furthermore, the user may input a position that is to serve as a reference through the input unit 1000. Information that the user inputs through the input unit 1000 in order to determine a specific position such as those mentioned above corresponds to information pertaining to the specific position.

It is to be noted that the exemplary embodiment is not limited to a mode in which the sampling frequencies are set individually for the respective acoustic wave receiving elements 300-1 through 300-8, and any technique that can reduce the amount of data associated with a specific frequency component in accordance with the shape of the subject can be employed.

For example, the sampling frequency determination unit 711 determines a sampling frequency on the basis of the distance L1_{—}a, which is the shortest among the distances from the center X of curvature to the surface of the subject E as viewed in the direction from the plurality of acoustic wave receiving elements 300 to the center X of curvature. Then, the sampling frequency determination unit 711 may set the sampling frequency determined on the basis of the distance L1_{—}a as a sampling frequency for each of the plurality of acoustic wave receiving elements 300. With the sampling frequency determined in this manner, at least a high-frequency component of a photoacoustic wave generated at the center X of curvature and reaching the acoustic wave receiving element 300-1 does not contribute to a reduction in the amount of data, and thus a decrease in image quality can be prevented.

The plurality of acoustic wave receiving elements 300 may be divided into several groups, and a sampling frequency may be assigned to each group. For example, acoustic wave receiving elements that are located at substantially equal distances from the subject or acoustic wave receiving elements that are located close to each other can be grouped together. For example, the acoustic wave receiving elements 300-1 and 300-2 that are located close to each other can form a group 1; the acoustic wave receiving elements 300-3 and 300-4 can form a group 2; the acoustic wave receiving elements 300-5 and 300-6 can form a group 3; and the acoustic wave receiving elements 300-7 and 300-8 can form a group 4. The method of forming the groups may be changed in accordance with the measurement positions of the support member 400 at the time of light irradiation. In this case, the grouping may be changed for each measurement position, or the grouping may be identical for a certain measurement position group.

A different sampling frequency may be set for each measurement position of the support member 400. Alternatively, the same sampling frequency may be set for a plurality of measurement positions.

The grouping or the setting of the sampling frequency may be changed when the measurement is carried out with a varied light irradiation mode even if the measurement position is identical.

Although a mode in which time-series reception signals are sampled at a constant sampling frequency has been described above, time-series reception signals outputted from the respective acoustic wave receiving elements may be sample at a sampling frequency that is varied in time series. Among the time-series reception signals, typically, a photoacoustic wave that is received at an earlier timing is a photoacoustic wave generated near the surface of the subject and thus does not attenuate to a great extent. In the meantime, typically, a photoacoustic wave that is received at a later timing is a photoacoustic wave generated at a portion deep inside the subject and thus attenuates to a great extent. In particular, a high-frequency component of a photoacoustic wave generated at a portion deep inside the subject attenuates to a greater extent than does a low-frequency component of the photoacoustic wave. Therefore, the sampling frequency determination unit 711 can reduce the sampling frequency for a reception signal, among the time-series reception signals, received at a later timing, and thus data of an attenuated high-frequency component can be selectively reduced.

In a case in which a constant sampling frequency is set for the time-series reception signals with the center of curvature serving as a reference position as in the example described above, a photoacoustic wave that is generated near the surface of the subject and that does not attenuate to a great extent may not be sampled with a high degree of fidelity. In other words, a high-frequency component that is generated near the surface of the subject and that has a high S/N ratio may not be sampled with a high degree of fidelity. On the other hand, as the sampling frequency is varied in time series, a frequency component having a sufficient S/N ratio is selectively stored at each receiving timing, and the amount of data can be reduced effectively.

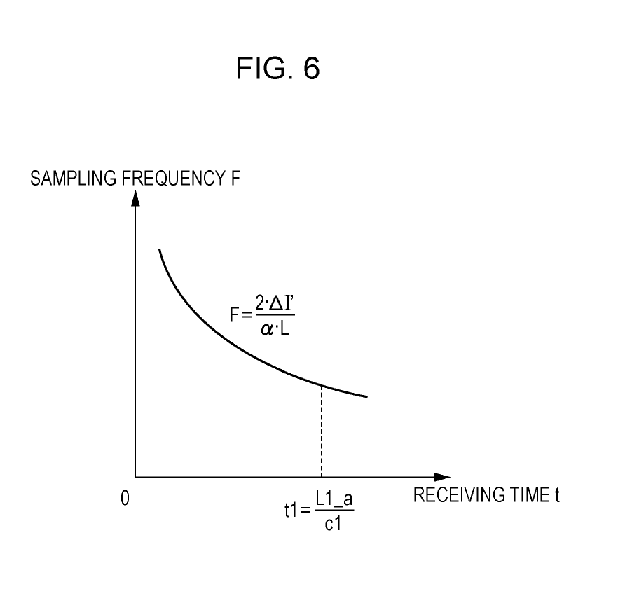

For example, a case in which the sampling frequency for the acoustic wave receiving element 300-1 illustrated in FIG. 3 is varied in time series will be considered. FIG. 6 illustrates an example of the sampling frequency for the acoustic wave receiving element 300-1. In FIG. 6, the horizontal axis represents the receiving time t, and the vertical axis represents the sampling frequency F. A timing at which a photoacoustic wave generated at the surface of the subject reaches the acoustic wave receiving element 300-1 is defined as the receiving time t=0. Here, the receiving time t corresponds to a value obtained by dividing the distance L from the center X of curvature to the surface of the subject E by the speed of sound c1 inside the subject E.

As described above, a photoacoustic wave generated at a portion deep inside the subject E attenuates to a greater extent than does a low-frequency component, and thus the low-frequency component becomes dominant. Therefore, in FIG. 6 as well, the sampling frequency F is reduced as the receiving time progresses or at a later receiving timing, so that a low-frequency component can be sampled selectively. In addition, in FIG. 6, the sampling frequency F is set to a value that is twice the frequency f determined through Expression (3). For example, a reception signal that corresponds to the receiving time t1=L1_{—}a/c1 of a photoacoustic wave generated at the center X of curvature is sampled at the sampling frequency F=2ΔI′/αL1_{—}a.

Attenuation of an acoustic wave at the receiving time t=0 cannot be conceived, and an acoustic wave at any frequency can be received. Thus, the initial value (F(0)) of the sampling frequency F may become infinite. In reality, however, an appropriate value that is no less than twice an upper limit of the frequency band targeted by the user can be set to F(0). F(0) being set as the initial value, the sampling frequency may be set to a value no less than the sampling frequency F indicated in FIG. 6 and less than F(0) as the receiving time progresses, and thus a reduction in the amount of data may be achieved.

Instead of changing the sampling frequency for each receiving time, a sampling frequency corresponding to a given receiving time may be set as a sampling frequency of another receiving time of a close timing. In other words, the sampling frequency may be varied stepwise in time series.

When the measurement position changes, the positional relationship of the acoustic wave receiving element and the subject may also change. Thus, a frequency component contained in a photoacoustic wave received by the acoustic wave receiving element may change in accordance with the measurement position. Therefore, if the sampling frequency is not changed when the measurement position is changed, the amount of data associated with a reception signal of a photoacoustic wave of a high-frequency component that could have been received at a high intensity may be reduced. Accordingly, the sampling frequency determination unit 711 determines the sampling frequencies for the plurality of acoustic wave receiving elements 300 on the basis of the information on the measurement positions and can thus determine the sampling frequencies that are appropriate for the respective measurement positions.

In addition, when the shape of the subject changes, the positional relationship of the acoustic wave receiving element and the subject may also change. Thus, a frequency component contained in a photoacoustic wave received by the acoustic wave receiving element may change in accordance with the shape of the subject. Therefore, if the sampling frequency is not changed when the shape of the subject is changed, the amount of data associated with a reception signal of a photoacoustic wave of a high-frequency component that could have been received at a high intensity may be reduced. Accordingly, the sampling frequency determination unit 711 determines the sampling frequencies for the plurality of acoustic wave receiving elements 300 on the basis of the information that is based on the shape of the subject and can thus determine the sampling frequency appropriate for the shape of the subject at the time of the measurement.

### S400: Process of Acquiring Reception-Signal Data by Sampling Time-Series Reception Signals at Determined Sampling Frequencies

The scanner 500 positions the support member 400 at one of the measurement positions set in S200. The CPU 731 outputs a control signal such that the light source 100 emits light when the support member 400 is positioned at the set measurement position. The light is guided by the optical system 200 and reaches the subject E through the acoustic matching material 1300. Then, the light that has reached the subject E is absorbed by the subject E, and a photoacoustic wave is generated.

The plurality of acoustic wave receiving elements 300 receive the photoacoustic wave that has been generated inside the subject E and has propagated through the acoustic matching material 1300 and converts the received photoacoustic wave to electric signals serving as the time-series reception signals.

Then, the signal data acquisition unit 710 samples the time-series reception signals at the sampling frequencies determined in S300 and stores the sampled data as the reception-signal data.

Hereinafter, with reference to the computer 700 illustrated in FIG. 5, a specific example of the method for sampling the reception signals at the sampling frequencies determined in S300 will be described.

The plurality of acoustic wave receiving elements 300-1 through 300-8 receive the photoacoustic wave, converts the received photoacoustic wave to electric signals, and outputs the electric signals to respective AD converters (ADCs) 717-1 through 717-8. The ADCs 717-1 through 717-8 sample the electric signals at a certain frequency in accordance with a clock outputted from a system CLK 713 so as to convert the electric signals to digital signals, and output the digital signals to respective first-in first-out memories (hereinafter, the FIFOs) 716-1 through 716-8. The FIFOs 716-1 through 716-8 store the digital signals outputted from the respective ADCs 717-1 through 717-8 in accordance with a clock outputted from the system CLK 713 and a write-enable outputted from a FIFO control unit 712.

In the signal data acquisition unit 710, information on the sampling frequencies that are determined in S300 and outputted from the sampling frequency determination unit 711 is inputted to the FIFO control unit 712 and the system CLK 713. The FIFO control unit 712 supplies write-enables [1] through [8] and read-enables [1] through [8] to the respective FIFOs 716-1 through 716-8. In addition, the system CLK 713 supplies sampling clocks [1] through [8] to the respective ADCs 717-1 through 717-8. Furthermore, the system CLK 713 supplies writing clocks [1] through [8] and reading clocks [1] through [8] to the respective FIFOs 716-1 through 716-8. The FIFO control unit 712 and the system CLK 713 control the mode of sampling the time-series reception signals outputted from the plurality of acoustic wave receiving elements 300 in accordance with the information on the sampling frequencies outputted from the sampling frequency determination unit 711.

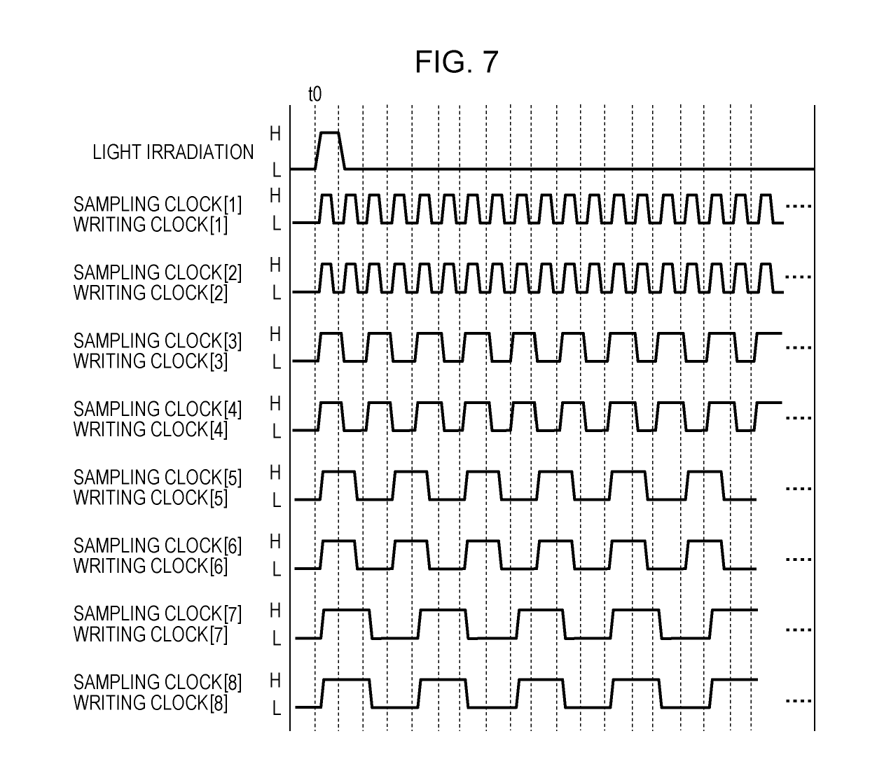

FIG. 7 illustrates the sampling clocks [1] through [8] and the writing clocks [1] through [8] that the system CLK 713 supplies, respectively, to the ADCs 717-1 through 717-8 and the FIFOs 716-1 through 716-8 in the measurement state illustrated in FIG. 3. In other words, FIG. 7 illustrates a sampling sequence that is based on the sampling frequencies determined in S300. FIG. 7 indicates that the ADCs 717-1 through 717-8 each carry out AD conversion when the level of the corresponding sampling clock changes from L to H but the ADCs 717-1 through 717-8 do not carry out AD conversion in other cases. In addition, FIG. 7 indicates that the writing into each of the FIFOs 716-1 through 716-8 is carried out when the level of the corresponding writing clock changes from L to H but the writing into the FIFOs 716-1 through 716-8 is not carried out in other cases.

For example, in the present exemplary embodiment, the sampling frequencies of the acoustic wave receiving element 300-1 through the acoustic wave receiving element 300-8 are set progressively lower on the basis of the sampling frequencies determined in S300.

The reception signals of the photoacoustic wave received by the acoustic wave receiving elements 300-1 and 300-2 are sampled at the sampling clocks [1] and [2] and the writing clocks [1] and [2] of the same frequency. The reception signals of the photoacoustic wave received by the acoustic wave receiving elements 300-3 and 300-4 are sampled at the sampling clocks [3] and [4] and the writing clocks [3] and [4] of the same frequency. The reception signals of the photoacoustic wave received by the acoustic wave receiving elements 300-5 and 300-6 are sampled at the sampling clocks [5] and [6] and the writing clocks [5] and [6] of the same frequency. The reception signals of the photoacoustic wave received by the acoustic wave receiving elements 300-7 and 300-8 are sampled at the sampling clocks [7] and [8] and the writing clocks [7] and [8] of the same frequency.

Subsequently, the FIFOs 716-1 through 716-8 transfer the stored reception-signal data to a dynamic random-access memory (DRAM) 718, which corresponds to a final-stage storage unit, in accordance with the clocks outputted from the system CLK 713 and the read-enables outputted from the FIFO control unit 712. A select switch 714 selects one of the FIFOs 716-1 through 716-8, connects the selected one to the DRAM 718, and transfers the digital signal to the DRAM 718. In this manner, the DRAM 718 stores a digital signal in which a reception signal corresponding to a high-frequency component has been reduced as the reception-signal data. As a reception signal corresponding to a high-frequency component is reduced in the data stored in the DRAM 718, the amount of data is reduced. Therefore, according to the present exemplary embodiment, the DRAM 718 does not require a memory capacity that allows the entire time-series reception signals to be stored therein, and thus the memory capacity of the DRAM 718 can be reduced. The DRAM 718 and a DRAM 722 may each be a storage medium of another type, such as a static random-access memory (SRAM) and a flash memory. Any storage medium may be used as such a storage medium as long as the capacity, the writing rate, and the readout rate that do not cause a problem in the system operation are ensured.

The reception-signal data as used in the present specification refers to time-series signal data that is to be used to acquire the subject information in the information acquisition unit 720, which will be described later. In other words, the reception-signal data refers to the time-series signal data that is stored in the final-stage storage unit or the DRAM 718 of the signal data acquisition unit 710. Therefore, according to the present exemplary embodiment, it is sufficient if the data stored in the final-stage storage unit of the signal data acquisition unit 710 has been acquired by sampling the reception signals at the sampling frequencies determined in S300.

The reception signals may be sampled at a predetermined sampling frequency when the reception signals are to be stored in an initial-stage storage unit, and the reception signals may then be sampled at the sampling frequencies determined in S300 when the reception signals are to be transferred to a later-stage storage unit from a storage unit of a preceding stage. In this case as well, the amount of data associated with the reception-signal data stored in the final-stage storage unit can be reduced.

In order to reduce the memory space in each of the storage units of the signal data acquisition unit 710, it is preferable that the amount of data stored in a preceding-stage storage unit be reduced as much as possible. In particular, it is preferable that the reception signals be sampled at the sampling frequencies determined in S300 before the reception signals are stored in the initial-stage storage unit or the FIFOs 716 of the signal data acquisition unit 710 so as to reduce the amount of data, as in the present exemplary embodiment. As the amount of data in a preceding-stage storage unit is reduced in this manner, the amount of data to be transferred to a later-stage storage unit can be reduced, and thus the time it takes to transfer the data can be reduced.

When the sampling frequency is changed in time series, it may be difficult to change the clock frequency of the ADCs 717. Therefore, the ADCs 717 may carry out AD conversion at a constant frequency and store digital signals in the FIFOs 716 serving as the initial-stage storage units. Then, the digital signals may be resampled at the sampling frequencies determined in S300 when the digital signals are transferred from the FIFOs 716 to a later-stage storage unit.

The sampling clocks may be set to a predetermined frequency f_{H}, and the write enables of the FIFOs 716 may be turned to the H level for one clock cycle with every N clock cycles. In the result, the sampling frequency may substantially be set to f_{H}/N. When N is varied over time, the sampling frequency can also be varied in time series.

The destination to which the digital signals that the initial-stage storage units have acquired are transferred is not limited to a later-stage storage unit. In other words, the digital signals that the initial-stage storage units have acquired may be outputted to an arithmetic unit, and the digital signals, having been subjected to preprocessing such as noise preprocessing in the arithmetic unit, may then be transferred to a later-stage storage unit.

It is preferable that the reception-signal data be associated with information, such as the positional information of the support member and the number of instances of light irradiation, and then be stored. For example, when the digital signals are transferred from the FIFOs 716-1 through 716-8 to the DRAM 718, a header or a trailer may be appended to the head or the end of the digital signal group. Examples of information contained in the header or the trailer include the numbers of the acoustic wave receiving elements with which the digital signal group has been acquired, the positional information of the support member, the number of instances of light irradiation, and a data amount reduction period. One or both of the header and the trailer may be provided. When the header and the trailer are both provided, to which one of the header and the trailer each piece of information is to be assigned may be determined as appropriate.

Control similar to the control according to the present exemplary embodiment can be achieved even when RAMs, instead of the FIFOs, are used.

In addition, in this process, processing for reducing the amount of data associated with a reception signal generated in a region other than the inside of the subject may also be carried out.

As long as a reception signal of a target frequency component can selectively be sampled as appropriate, the reception-signal data may be acquired through any technique from the time-series reception signals outputted from the respective acoustic wave receiving elements 300.

S500: Process of Determining Whether Reception-Signal Data has been Acquired at Entire Measurement Positions

Subsequently, the CPU 731 determines whether the reception-signal data has been acquired at the entire measurement positions set in S200. If the reception-signal data has not been acquired at the entire measurement positions, the processing returns to S400. Specifically, the CPU 731 moves the support member 400 to a subsequent measurement position with the scanner 500 and causes the photoacoustic apparatus to execute the process of acquiring the reception-signal data as described in S400.

In this manner, as S400 is repeated at the respective measurement positions, the amount of data associated with the reception signals in the data amount reduction period corresponding to each measurement position can be reduced.

### S600: Process of Acquiring Subject Information on the Basis of Reception-Signal Data

The information acquisition unit 720 acquires the subject information on the basis of the reception-signal data acquired in S400. Specifically, a GPU 721 of the information acquisition unit 720 carries out a process that is based on an image reconstruction algorithm on the reception-signal data stored in the DRAM 718 so as to acquire the subject information and stores the subject information in the DRAM 722.

As described above, the reception-signal data acquired in S400 is data corresponding to, of a photoacoustic wave generated inside the subject, a frequency component of the photoacoustic wave that has reached the acoustic wave receiving elements at a high intensity. Therefore, in this process, the subject information having a high S/N ratio can be acquired, as compared with a case in which the subject information is acquired by using a frequency component of the photoacoustic wave having a low intensity.

This process may be carried out between S400 and S500. Specifically, the subject information may be acquired successively on the basis of the reception-signal data acquired when the support member 400 is located at the respective measurement positions. In this case, it is preferable that a single piece of subject information be generated by combining a plurality of pieces of subject information, acquired successively, corresponding to the respective positions of the support member 400 by adding or averaging the plurality of pieces of subject information. In this manner, the subject information can be acquired on the basis of the reception-signal data acquired at at least one measurement position before the reception-signal data at the entire measurement positions is acquired, and thus the time it takes to acquire the subject information that is based on the entire pieces of reception-signal data can be reduced.

### S700: Process of Displaying Subject Information

The display 900 displays the subject information acquired in S600 in the form of a distribution image or numeric data. For example, the CPU 731 reads out the subject information from the DRAM 722 and displays the distribution image of the subject information on the display 900.

As described thus far, the photoacoustic apparatus according to the present exemplary embodiment can set the sampling frequencies for selectively sampling the reception signals of a high-intensity photoacoustic wave reaching the plurality of acoustic wave receiving elements 300. Through this configuration, the amount of data associated with the reception signals of an attenuated frequency component contained in the photoacoustic wave can selectively be reduced. In other words, the reception signals that contribute to acquiring the subject information having a high S/N ratio can selectively be acquired. Accordingly, the amount of data associated with an attenuated frequency component contained in the photoacoustic wave can be reduced, and thus the memory space for storing the reception-signal data can be reduced.

The data amount reduction period in the exemplary embodiments may be set based on the distance or the time. Alternatively, the data amount reduction period may be set based on the sampling clock count of the ADCs, the system CLK count, or the data count. In addition, the data amount reduction period may be set by any means that can specify a region.

In addition, although an example in which the number of the acoustic wave receiving elements is eight has been illustrated in the exemplary embodiments, the number of the acoustic wave receiving elements is not limited to such an example. The acoustic wave receiving elements may be provided in any number in accordance with the specification of the photoacoustic apparatus.

The timing at which the data acquisition period ends may be set at the same timing for the entire acoustic wave receiving elements or may be set individually for each of the acoustic wave receiving elements.

In a case in which the timing at which the data acquisition period ends is set individually for each of the acoustic wave receiving elements, a region on the directional axis in which the subject is not present may be determined for each of the acoustic wave receiving elements on the basis of the shape information of the subject, and the determination result may be reflected on the timing at which the data acquisition period ends.

### OTHER EMBODIMENTS

Additional embodiment(s) can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to exemplary embodiments, it is to be understood that these exemplary embodiments are not seen to be limiting. The scope of the following claims is to be accorded the broadest interpretation so as to encompass all such modifications and equivalent structures and functions.

This application claims the benefit of Japanese Patent Application No. 2014-100853, filed May 14, 2014, which is hereby incorporated by reference herein in its entirety.

What is claimed is:

1. A photoacoustic apparatus, comprising:
a light source;
a plurality of receiving elements configured to receive a photoacoustic wave generated as a subject is irradiated with light emitted from the light source and output time-series reception signals;
a signal data acquisition unit configured to generate reception-signal data based on the time-series reception signals and store the reception-signal data; and
an information acquisition unit configured to acquire information on the subject based on the reception-signal data stored in the signal data acquisition unit,
wherein the signal data acquisition unit determines a sampling frequency based on a distance from a specific position to a surface of the subject, samples the time-series reception signals at the sampling frequency so as to generate the reception-signal data, and stores the reception-signal data.

2. The photoacoustic apparatus according to claim 1,
wherein the signal data acquisition unit determines the sampling frequency at which a photoacoustic wave of a component having a frequency f expressed through the following expression can be sampled,
$$f = \frac{Δ {I}^{′}}{α \cdot L}$$
wherein α is a frequency-dependent attenuation of the subject, ΔI′ is a permissible attenuation, and L is the distance.

3. The photoacoustic apparatus according to claim 2,
wherein the signal data acquisition unit determines a frequency that is no less than twice the frequency f and no greater than 10 times the frequency f as the sampling frequency.

4. The photoacoustic apparatus according to claim 3,
wherein the signal data acquisition unit determines a frequency that is no less than twice the frequency f and no greater than four times the frequency f as the sampling frequency.

5. The photoacoustic apparatus according to claim 2, further comprising:
an input unit configured to receive an input of the permissible attenuation.

6. The photoacoustic apparatus according to claim 1, further comprising:
an input unit configured to receive an input of information pertaining to the specific position.

7. The photoacoustic apparatus according to claim 1, further comprising:
a support member configured to support the plurality of receiving elements,
wherein the support member supports the plurality of receiving elements such that directional axes of at least some of the plurality of receiving elements converge, and
wherein the signal data acquisition unit sets a position at which the directional axes of the at least some of the plurality of receiving elements converge as the specific position.

8. The photoacoustic apparatus according to claim 1, further comprising:
a support member configured to support the plurality of receiving elements,
wherein the support member has a shape that is a sphere-based shape, and
wherein the signal data acquisition unit sets a center of curvature of the support member as the specific position.

9. The photoacoustic apparatus according to claim 1,
wherein the signal data acquisition unit determines the sampling frequency that varies in time series, samples the time-series reception signals at the sampling frequency that varies in time series so as to generate the reception-signal data, and stores the reception-signal data.

10. The photoacoustic apparatus according to claim 9,
wherein the signal data acquisition unit reduces the sampling frequency as a receiving timing of the time-series reception signals progresses.

11. The photoacoustic apparatus according to claim 1,
wherein the signal data acquisition unit
includes a first storage unit and a second storage unit,
samples the time-series reception signals outputted from the receiving elements into digital signals and stores the digital signals in the first storage unit, and
samples the digital signals stored in the first storage unit at the sampling frequency so as to generate the reception-signal data and stores the reception-signal data in the second storage unit.

12. The photoacoustic apparatus according to claim 1,
wherein the signal data acquisition unit samples the time-series reception signals outputted from the respective receiving elements at different sampling frequencies so as to generate the reception-signal data and stores the reception-signal data.

13. The photoacoustic apparatus according to claim 1, further comprising:
a support member configured to support the plurality of receiving elements,
wherein the support member supports the plurality of receiving elements such that directional axes of at least some of the plurality of receiving elements converge.

14. A photoacoustic apparatus, comprising:
a light source;
a plurality of receiving elements configured to receive a photoacoustic wave generated as a subject is irradiated with light emitted from the light source and output time-series reception signals;
a signal data acquisition unit configured to generate reception-signal data based on the time-series reception signals and store the reception-signal data; and
an information acquisition unit configured to acquire information on the subject based on the reception-signal data stored in the signal data acquisition unit,
wherein the signal data acquisition unit determines a sampling frequency at which, from among frequency components of a photoacoustic wave generated at a specific position, a frequency component whose attenuation is no greater than a permissible attenuation can be sampled and a frequency component whose attenuation is greater than the permissible attenuation cannot be sampled, samples the time-series reception signals at the sampling frequency so as to generate the reception-signal data, and stores the reception-signal data.

15. A photoacoustic apparatus, comprising:
a light source;
a plurality of receiving elements configured to receive a photoacoustic wave generated as a subject is irradiated with light emitted from the light source and output time-series reception signals;
a signal data acquisition unit configured to generate reception-signal data in which an amount of data associated with the time-series reception signals is reduced and store the reception-signal data; and
an information acquisition unit configured to acquire information on the subject based on the reception-signal data stored in the signal data acquisition unit,
wherein the signal data acquisition unit samples the time-series reception signals outputted from the respective receiving elements at different sampling frequencies so as to generate a plurality of pieces of reception-signal data and stores the plurality of pieces of reception-signal data.
